(19) United States
(12) Patent Application Publication
(10) Pub. No.: US 2020/0360576 A1
(43) Pub. Date: Nov. 19, 2020

(54) SYSTEMS AND METHODS THAT FACILITATE TATTOO REMOVAL

(71) Applicant: THE GENERAL HOSPITAL CORPORATION, Boston, MA (US)

(72) Inventors: Lilit Garibyan, Boston, MA (US); Richard Rox Anderson, Boston, MA (US); William Farinelli, Boston, MA (US); Fernanda H. Sakamoto, Boston, MA (US); Martin Purschke, Boston, MA (US)

(21) Appl. No.: 16/944,588

(22) Filed: Jul. 31, 2020

Related U.S. Application Data
(63) Continuation-in-part of application No. 16/069,339, filed on Jul. 11, 2018.
(60) Provisional application No. 62/278,014, filed on Jan. 13, 2016.

(51) Int. Cl. A61M 1/00

(52) U.S. Cl. CPC A61M 1/009; A61M 2210/04; A61M 2202/04; A61M 1/0062

(57) ABSTRACT

After a tattoo removal process fragments of the tattoo (pigment particles) may be left in the patient's skin. Pressure can be applied to the patient's skin to move the pigment particles deeper within the patient's skin. The pigment particles can become less visible when they are deeper within the patient's skin.

### RELATED APPLICATIONS

This application is a continuation in part of U.S. Non-Provisional application Ser. No. 16/069,339, entitled “SYSTEMS AND METHODS FOR REMOVING EXOGENOUS PARTICLES FROM THE SKIN OF A PATIENT,” filed 11 Jul. 2018, which is a National Stage entry of Serial No. PCT/US2017/013548, entitled “SYSTEMS AND METHODS FOR REMOVING EXOGENOUS PARTICLES FROM THE SKIN OF A PATIENT,” filed 13 Jan. 2017, which claims the benefit of U.S. Provisional Application No. 62/278,014, entitled “SYSTEMS AND METHODS FOR REMOVING EXOGENOUS PARTICLES FROM THE SKIN OF A PATIENT,” filed Jan. 13, 2016. The entirety of these applications is hereby incorporated by reference for all purposes.

### TECHNICAL FIELD

The present disclosure relates generally to tattoo removal and, more particularly, to systems and methods that can be used to reduce the visibility of a tattoo after a tattoo removal process.

### BACKGROUND

Tattoos are made by inserting ink or other colorant into the dermis of the skin. More than forty million people in the United States have tattoos, and many people will seek to have them removed. While tattoos are considered permanent, it is sometimes possible to fully or partially remove tattoos. Current tattoo removal technologies involve laser treatments (e.g., using Q-switched lasers, picosecond lasers, or the like). To remove the tattoo completely, a patient may be required to undergo a series of painful laser treatments (e.g., requiring 10-20 sessions). Even with multiple sessions, the laser treatments can leave tattoo pigments remaining in the skin and/or create textural changes to the skin, including scarring. Accordingly, many patients are unwilling to have their tattoo removed (even though they no longer want the tattoo) due to the associated time, cost and/or pain.

### SUMMARY

The present disclosure relates to reducing the visibility of a tattoo after a tattoo removal process. Systems and methods described herein can reduce the time, cost, and/or pain associated with tattoo removal.

In one aspect, the present disclosure can include a method reducing the visibility of a tattoo after a tattoo removal process. Pressure can be applied to the patient's skin to move the pigment particles deeper within the skin, making the tattoo particles less visible.

In another aspect, the present disclosure can include a system that can facilitate tattoo removal by reducing the visibility of a tattoo after a tattoo removal process. The system can include a pressure application device that can be configured to apply a pressure to a patient's skin to move freed pigment particles after a procedure that frees ink of a tattoo from cells to create the freed pigment particles within a patient's skin deeper within the patient's skin. The freed pigment particles can become less visible when they are deeper within the patient's skin.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram illustrating an example of a system that can reduce the visibility of a tattoo after a tattoo removal process according to an aspect of the present disclosure;

FIG. 2 is a block diagram illustrating another example of the system shown in FIG. 1;

FIG. 3 is a diagram illustrating an example of a wearable device;

FIG. 4 represents a top view indicating alternating pressure (o) and suction (x) that can be applied by the example wearable device of FIG.;

FIG. 5 is a process flow diagram illustrating a method for reducing the visibility of a tattoo after a tattoo removal process according to an aspect of the present disclosure; and

FIG. 6 is a process flow diagram illustrating a method for dermal lavage that can be used with the method of FIG. 5.

### DETAILED DESCRIPTION

### Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “tattoo” can refer to a design created by inserting pigment into the dermis of a subject's skin. In many cases, the tattoo is intended to be permanent.

As used herein, the term “tattoo removal” can refer to a procedure done to try to remove at least a portion of an unwanted tattoo. Common tattoo removal techniques include laser removal (e.g., using Q-switched lasers, picosecond lasers, or the like), dermabrasion, TCA, salabrasion, cryosurgery, excision, or the like.

As used herein, the term “exogenous” can refer to an element or substance that is placed into the body originating from an external source (e.g., not natural to the body). Accordingly, the term “exogenous particle” can refer to a particle that is placed into the body from an external source. In some instances, an exogenous particle can be an ink pigment that is placed in the skin of a patient. For example, the ink pigment can be a remnant of a tattoo created by a laser removal process.

As used herein, the term “endogenous” can refer to an element or substance that is placed into the body originating from a source within the body (e.g., a cell. a tissue, or an organism).

As used herein, the term “skin” can refer to the soft outer covering of vertebrates, including the epidermis (e.g., the outermost layer of cells in the skin) and the dermis (e.g., a layer of skin between the epidermis and subcutaneous tissue that cushions the body from stress and strain).

As used herein, the term “subcutaneous” refers to cells and/or tissues located beneath the skin.

As used herein, the terms “target region” and “target area” can be used interchangeably and can refer to an area of a patient's skin from which a tattoo needs to be removed. The target region can include particles of ink left over after a tattoo removal process.

As used herein, the term “irrigation area” can refer to an area of a patient's skin that will be irrigated to remove particles. Of ink left over after a tattoo removal. In some instances, the irrigation area can be less than, greater than, or equal to the target area.

As used herein, the term “conduit” can refer to an element, structure, or component for conveying a fluid. The conduit can be internal (e.g., needles, microneedels, or the like) and/or external (e.g., a container housing the solution) to a patient's skin. Additionally, when the conduit refers to an external conduit, the conduit can be in contact with the patient's skin (e.g., a component used for electrophoresis, iontophoresis, or the like) or not in contact with the patient's skin.

As used herein, the term “channel” can refer to a passageway or conduit extending from an opening or hole at the surface of the skin to a depth under the skin.

As used herein, the term “microchannel” can refer to a channel with a diameter less than about 1 mm. For example, a microchannel can be a channel that extends from an opening or hole in a patient's skin, through the epidermis, and into the dermis. As an example, a microchannel can be created in a patient's skin by a component of a fluid removal device, such as needle, a laser, or the like.

As used herein, the term “lavage” can refer to washing or irrigating with one or more injections of a fluid (e.g., water, saline, or other biocompatible fluid, which may include a pharmaceutical agent and/or an enzyme and/or any natural products or toxins, such as spider venom, snake venom, beetle venom, bee venom, or the like).

As used herein, the term “pressure” can refer to a mechanical force exerted on or against an object by something in contact with the object. In some instances, pressure can be a continuous physical force. In other instances, pressure can be vibration, suction, or the like.

As used herein, the terms “subject” and “patient” can be used interchangeably and refer to any warm-blooded living organism including, but not limited to, a human being, a pig, a rat, a mouse, a dog, a cat, a goat, a sheep, a horse, a monkey, an ape, a rabbit, a cow, etc.

### Overview

The present disclosure relates generally to systems and methods for tattoo removal. Although tattoos are generally intended to be permanent, after a time, many individuals want to remove their tattoo(s). Accordingly, tattoo removal procedures, including laser removal (e.g., using Q-switched lasers), dermabrasion, TCA, salabrasion, cryosurgery, excision, or the like, can be used to remove at least a portion of an unwanted tattoo. However, the tattoo removal procedures can only remove a portion of the tattoo. Often, thousands of microscopic tattoo particles are released from cells in the dermis of the skin. While some of the tattoo particles are washed away by the patient's lymphatic system, other tattoo particles remain in the skin. The remaining tattoo particles, which are engulfed by macrophages and other phagocytes in the skin, essentially preserve the tattoo image. To remove these remaining tattoo particles completely, further laser removal treatments are required.

Advantageously, the present disclosure describes systems and methods that can reduce or eliminate the need for further laser removal treatments by reducing the visibility of a tattoo after a tattoo removal process. As discussed in more detailed below, the present disclosure provides systems and methods that can be used after a tattoo removal process to remove the remaining particles by applying pressure to the patient's skin to move the pigment particles deeper within the skin, making the tattoo particles less visible. In some instances, dermal lavage can also be used (e.g., before application of the pressure, after application of the pressure, and/or during application of the pressure) to facilitate removal of the tattoo particles.

### Systems

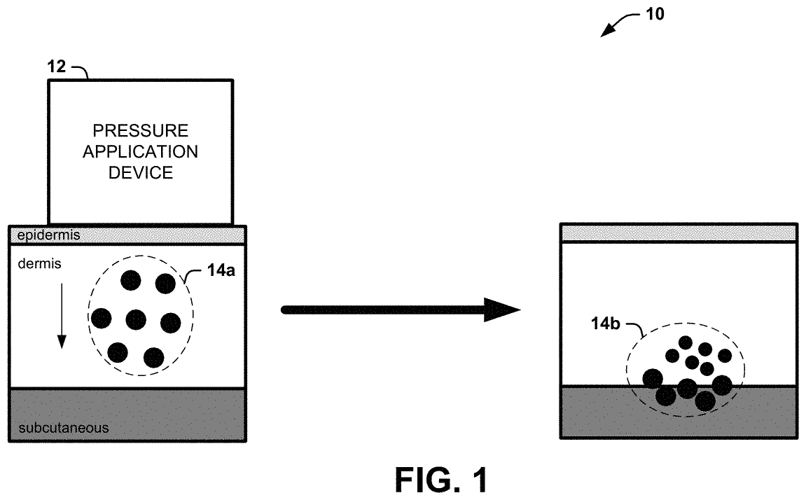

One aspect of the present disclosure can include a system 10 (FIG. 1) that can reduce the visibility of a tattoo after a tattoo removal process according to an aspect of the present disclosure. The tattoo removal process can include tattoo removal procedures, including laser removal (e.g., using Q-switched lasers, picosecond lasers, or the like), dermabrasion, TCA, salabrasion, cryosurgery, excision, or the like. After the tattoo removal process, particles of pigment (created by the tattoo removal process) can be left within the patient's skin. While some of the particles of pigment can be washed away by the patient's lymphatic system, other particles remain in the skin, are engulfed by macrophages and other phagocytes, and preserve the tattoo image, creating a residual tattoo. It should be noted that the tattoo removal process can also include removing one or more layers of the skin (e.g., the epidermis). For example, a suction blister can be created to remove one or more layers of skin. In another example, a fractional laser (e.g., an ablative laser, such as an erbium ablative laser or a CO₂ ablative laser) can be used to remove one or more layers of skin. The layers of skin can be removed without scarring (e.g., by use of coring needles or a laser) to improve conventional tattoo removal (e.g., 10% ablation can lead to 10% of the skin/tattoo being removed).

A pressure application device 12, shown in FIG. 1, can be used to apply a force (which can be a pressure, a suction, a vibration, or the like) to the patient's skin. As an example, the pressure application device 12 can apply at least one type of pressure to the patient's skin (to an area above the tattoo), which may be applied as a shock wave, resulting in a sharp change of pressure in a narrow region (e.g., the region of at least a portion of the leftover pigment particles 14a). The pressure can be positive pressure, for example. As another example, the pressure application device 12 can apply a pattern of positive pressure and/or negative pressure (e.g., in an oscillating fashion), such as by a controllable oscillating positive and negative pressure device, with a shock wave of positive pressure. The controllable oscillating positive and negative pressure device can be any device that generates alternating pressure. Non-limiting examples of the controllable oscillating positive and negative pressure device include a breast pump, two solenoid valves, one supplying the positive pressure and the other supplying the negative pressure, with a programmable device controlling the oscillation cycle, a breast pump, etc. As a further example, the pressure application device can apply vibration to provide the pressure with a shock wave of positive pressure. The pressure application device 12, as another example, can apply any type of mechanical force that includes a shock wave.

The leftover pigment particles 14a, when exposed to the pressure (e.g., the shock wave of positive pressure), can be forced to move from the dermis toward/into the subcutaneous region (pigment particles 14b). The pigment particles 14b, which are closer to/within the subcutaneous tissue, make the tattoo less visible to someone looking at the patient. In other words, the pigment particles 14a are forced deeper into the skin an even into subcutaneous tissue such that pigment particles 14b no longer preserve as much of the tattoo image.

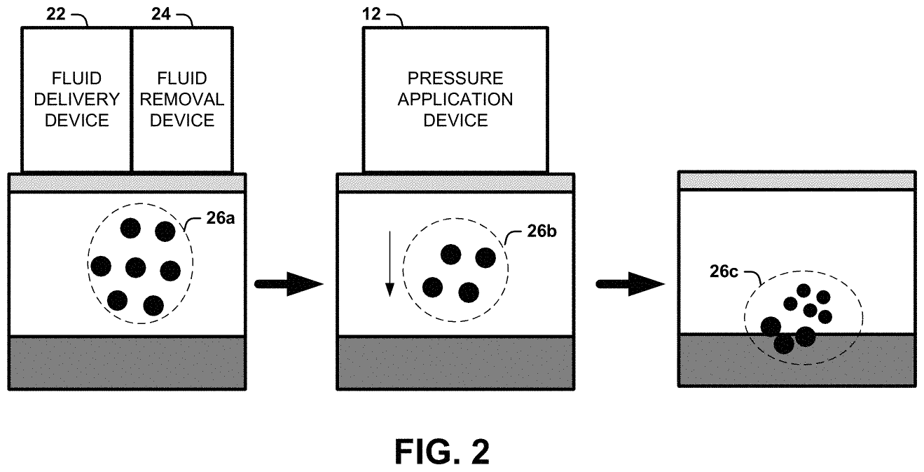

Although FIGS. 1 and 2 show an intact epidermis, at least a portion of the epidermis may be removed or disturbed before the pressure application device 12 applies the force. As previously noted, the pressure can be applied when a portion of one or more layers of the skin (e.g., the epidermis) has been removed. For example, a suction blister can be created to remove one or more layers of skin. In another example, a fractional laser (e.g., an ablative laser, such as an erbium ablative laser or a CO₂ ablative laser) can be used to remove one or more layers of skin. The layers of skin can be removed without scarring (e.g., by use of coring needles or a laser). The pressure can also be applied when at least a portion of the skin could additionally or alternatively be perforated with one or more needles (e.g., using a portion of a fluid deliver device 22 and/or a fluid removal device 24, shown in FIG. 2). It will be appreciated that at least a portion of the perforations can be treated with an anti-clotting drug, like heparin, aspirin, an NSAID, or other anticoagulant to prevent the closure of the channels.

A dermal lavage can be utilized to remove a portion of the pigment particles. The dermal lavage can inject an irrigation fluid into an irrigation area so that the fluid and the tattoo particles are removed from the skin (e.g., via one or more holes or channels formed in the target area of the skin). In some instances, the dermal lavage can be utilized between the tattoo removal process and the pressure application (as shown in FIG. 2). In other instances, the dermal lavage can be utilized after the pressure application. In further instances, the dermal lavage can be utilized in combination with (e.g., during) the pressure application. In still other instances, the dermal lavage can be utilized two or more of before, during, and after the pressure application.

As shown in FIG. 2, the dermal lavage can involve a fluid delivery device 22 and a fluid removal device 24 (which may be the same device and/or one or more may be part of a device with the pressure application device 12. The dermal lavage works by the fluid delivery device 22 delivering a fluid into an irrigation area and the fluid removal device 24 removing the fluid and a portion of the tattoo particles from the irrigation area.

The fluid delivery device 22 can deliver an irrigation fluid into the irrigation area. At least a portion of the tattoo particles in the irrigation area can be suspended in the irrigation fluid as a result. The fluid removal device 24 can remove at least a portion of the tattoo particles (e.g., 10%, 20%, 30%, 40%, 50%, 60%, 70%, 80%, 90%, 99% or more) in the irrigation area.

In some instances, at least a portion of fluid delivery device 22 (e.g., a perforated conduit, one or more microneedles, one or more coring needles, or the like in combination with a needle, a catheter, a cannula, or the like) can be inserted into or around the target region to define an irrigation area (including at least a portion of the tattoo particles). An irrigation fluid can be injected into the irrigation area via the fluid conduit. A fluid removal device 24 can be configured to remove the fluid from the irrigation area. For example, the fluid removal device 24 form one or more channels in the irrigation area so that at least a portion of the tattoo particles are removable from the irrigation area (e.g., via suction or passive diffusion) along with the irrigation fluid.

The fluid delivery device 22 can be operatively coupled to the fluid source to recite the irrigation fluid. The irrigation fluid can be a bolus of liquid (e.g., in the range of about 10-50 mL), which is injected into the irrigation area to the target area to be irrigated. In one example, the fluid source can be a reservoir for the irrigation fluid (e.g., an IV bag).

The irrigation fluid can be an exogenous fluid and/or an endogenous fluid. In some instances, the irrigation fluid can be an exogenous fluid, such as a biocompatible fluid, such as saline, sterile water, or the like. In other instances, the irrigation fluid can include an enzyme, natural compound, and/or a pharmaceutical agent. The enzyme or pharmaceutical agent can at least one of lyse cells, make the skin more porous (e.g., by breaking up the tight connections between collagen fibers in the dermis), prevent or inhibit phagocytosis, or otherwise facilitate removal of the irrigation fluid. Examples of enzymes or pharmaceutical agents that can be within the irrigation fluid include hyuronidase, collegenase, phospholipase, cantherone, colchicine, podophylin, hyaluronic acid, pederine, and sucrose. Examples of a natural compound (e.g., a venom or poison) that can be included in the irrigation fluid include natural or diluted forms of venoms, such as spider, snake, beetle, or bee. In other instances, the enzyme or other chemical agent can be injected before or after the exogenous fluid. For example, the target area can undergo a pre-treatment with histamine, lipase, and/or phospholipase, causing the target area to undergo an inflammatory reaction, which can induce edema and fluid collection/flow to improve draining of the tattoo particles.

In other instances, the fluid can be an endogenous fluid (e.g., derived or obtained from the patient's body). For example, the endogenous fluid can come from local dermal edema. The patient can be injected with a drug, such as a histamine, that is known to cause local dermal edema in the target region of the skin. The fluid can leave the target region through holes or channels, thereby removing the edema and, in the process, washing out the tattoo particles.

In some instances, the fluid removal device 24 can include a channel formation device that can be configured to create one or more holes or channels that extend through a portion of the irrigation area. In some instances, the channel formation device can include one or more microneedles. In other instances, the channel formation device can include a laser or an array of a plurality of lasers. In further instances, the channel formation device 16 can be a tattoo gun without any ink (“reverse tattooing”). In one example, the channel formation device is configured to form one or more holes or channels, each of which extends through the epidermis into the dermis. Holes or channels formed by the channel formation device can each have a hydraulic diameter of about 10 mm or less, about 5 mm or less, or about 1 mm or less. In some instances, holes or channels formed by the channel formation device 16 can be microchannels (e.g., having a diameter of at least about 500 μm). In other instances, an anti-clotting drug, like heparin, aspirin, an NSAID, or other anticoagulant can be used to prevent the closure of the holes or channels formed by the channel formation device.

Irrigation fluid, in some instances containing a pharmaceutical agent and/or an enzyme, can be injected into the skin and flow into the one or more holes or channels and then exit the skin with the suspended exogenous particles. In some instances, fluid flow through the holes(s) or channel(s) happens automatically when the hole(s) or channel(s) is/are created in the skin. In other instances, fluid flow occurs upon application of an external stimulus to the target area (e.g., pressure on the surface of the skin by a gloved finger of a medical professional). In still other instances, fluid flow occurs upon application of suction or negative pressure (e.g., from a suction device, such as a breast pump, a wound vacuum, or the like) to the target region. Prior to applying suction, a mechanical force (e.g., a vibration or shock wave) can be applied to the target region to break up attachment of the exogenous particles from the surrounding tissue and make the particles more mobile. As an example, a vibrator can be used as a tool to loosen the exogenous particles. Alternatively, the mechanical force(s) can be applied to the target region following laser therapy (but before formation of the holes or channels) and/or at any point prior to applying suction to the target region.

Suction can be applied continuously or intermittently. Alternatively, suction can be applied in a cyclic manner along with positive pressure to generate a massaging action on the skin and thereby facilitate migration of the exogenous particles to the skin surface. In some instances, the intensity of the suction can be changed over time and thus be applied as a gradient of increasing or decreasing negative pressure.

The fluid delivery device 22, the fluid removal device 24 and/or the pressure application device 12 can be embodied in a wearable device. In a simple example, the pressure application device 12 can be included within in a wearable device to supply the force (e.g., a positive force, which may be delivered as a shock wave) at one or more times. As another example, a wearable device can include the fluid delivery device 22, the fluid removal device 24, and the pressure application device 12 to perform the dermal lavage and the pressure application in a single device. As a further example, a wearable device can include the fluid delivery device 22 and the fluid removal device 24 to perform the dermal lavage. In any example, the wearable device can include one or more dissolvable components, one or more power sources/batteries, an attachment mechanism—like an adhesive mechanism) and/or one or more additional components.

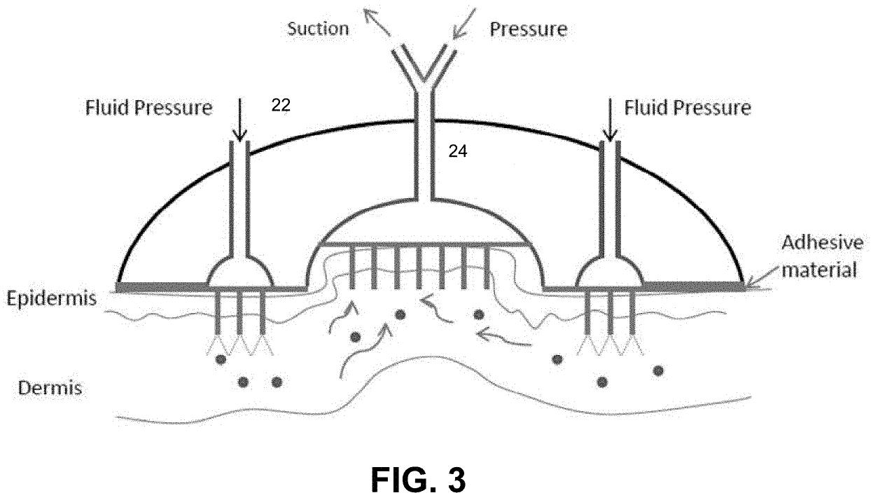

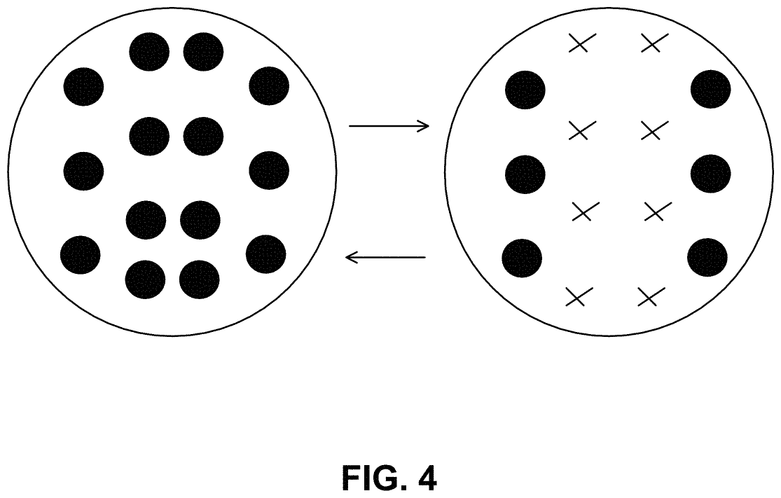

FIG. 3 shows an example of a wearable device to aid in the removal of the tattoo particles (e.g., the dermal lavage including the fluid delivery device and the fluid removal device 24). The device is held tightly to the skin with an adhesive patch. Small conduits can enter the skin and pressurized fluid could pass through them. The center chamber allows alternating suction (o) and pressure (x) (shown in FIG. 4) to flow such that when suction is on, the skin is pulled into the chamber and small conduits pierces the skin and allows suction (o) within the skin to occur, simultaneously causing fluid flow through. After a time, pressure (+) is forced into the chamber and pushes down on to the skin causing movement of the tattoo particles and flow through the conduits stops. This cycle continues in an alternating pattern. Suction and pressure times could be equal or suction time could be longer then pressure time. In some instances, after the dermal lavage, for example, a positive pressure (e.g., a shock wave) can be delivered into the skin at all locations to push remaining tattoo ink towards/into the subcutaneous tissue.

### Methods

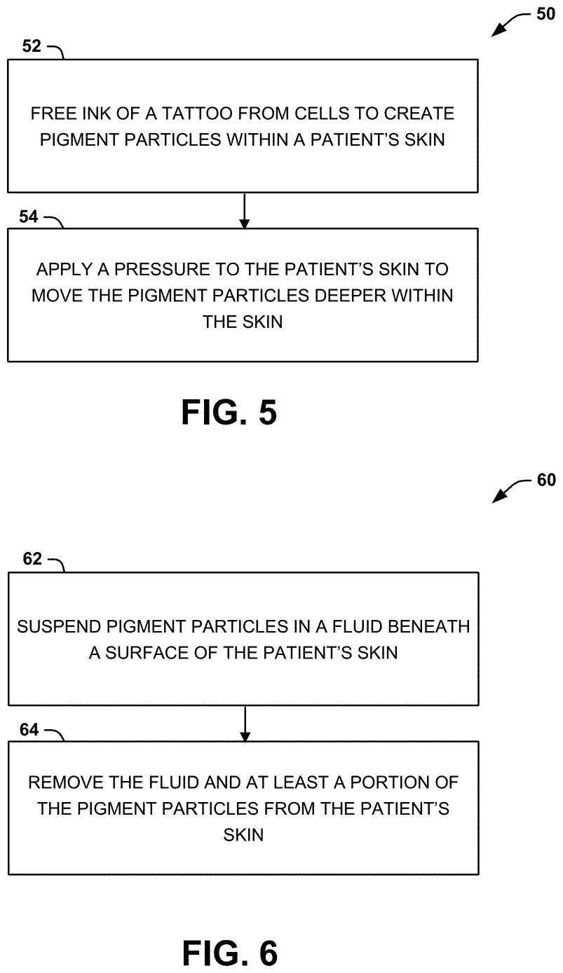

Another aspect of the present disclosure can include methods 50, 60 for reducing the visibility of a tattoo after a tattoo removal process, shown in FIGS. 5 and 6, which are illustrated as process flow diagrams. For purposes of simplicity, the methods 50, 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50 and 60. The methods 50 and 60 will be explained with regard to FIGS. 1-4, which illustrate systems that can be used to reduce the visibility of a tattoo after a tattoo removal process. It should be noted that at least a portion of the components executing the methods 50, 60 may be embodied in a wearable device.

Referring now to FIG. 5, illustrated is a method 59 for reducing the visibility of a tattoo after a tattoo removal process. At 52, ink of a tattoo can be freed from cells to create pigment particles within a patient's skin (during the tattoo removal process). The tattoo removal process can include tattoo removal procedures, including laser removal (e.g., using Q-switched lasers), dermabrasion, TCA, salabrasion, cryosurgery, excision, or the like. After the tattoo removal process, particles of pigment (created by the tattoo removal process) can be left within the patient's skin. While some of the particles of pigment can be washed away by the patient's lymphatic system, other particles remain in the skin, are engulfed by macrophages and other phagocytes, and preserve the tattoo image, creating a residual tattoo. It should be noted that the tattoo removal process can also include removing one or more layers of the skin (e.g., the epidermis). For example, a suction blister can be created to remove one or more layers of skin. In another example, a fractional laser (e.g., an ablative laser, such as an erbium ablative laser or a CO2 ablative laser) can be used to remove one or more layers of skin. The layers of skin can be removed without scarring (e.g., by use of coring needles or a laser) to improve conventional tattoo removal (e.g., 10% ablation can lead to 10% of the skin/tattoo being removed).

At 54, a pressure can be applied to the patient's skin (e.g., by pressure application device 12) to move the pigment particles deeper within the patient's skin so that the pigment particles become less visible. As an example, the pressure can be applied apply as a positive pressure to the patient's skin (to an area above the tattoo), which may be applied as a shock wave, resulting in a sharp change of pressure in a narrow region (e.g., the region of at least a portion of the leftover pigment particles 14a). As another example, pressure can be applied as a positive pressure and/or negative pressure (e.g., in an oscillating fashion), such as by a breast pump, with a shock wave of positive pressure. As a further example, the pressure can be applied as vibration, suction, or other mechanical force to provide the pressure, which can include a shock wave. The leftover pigment particles, when exposed to the pressure (e.g., the shock wave of positive pressure), can be forced to move from the dermis toward/into the subcutaneous region. The pigment particles, which are closer to/within the subcutaneous tissue, make the tattoo less visible to someone looking at the patient.

As previously noted, the pressure can be applied when a portion of one or more layers of the skin (e.g., the epidermis) has been removed. For example, a suction blister can be created to remove one or more layers of skin. In another example, a fractional laser (e.g., an ablative laser, such as an erbium ablative laser or a CO₂ ablative laser) can be used to remove one or more layers of skin. The layers of skin can be removed without scarring (e.g., by use of coring needles or a laser). The pressure can also be applied when at least a portion of the skin could additionally or alternatively be perforated with one or more needles. It will be appreciated that at least a portion of the perforations can be treated with an anti-clotting drug, like heparin, aspirin, an NSAID, or other anticoagulant to prevent the closure of the channels.

Referring now to FIG. 6, illustrated is a method 60 for performing a dermal lavage, which can be used in connection with the method 50 (e.g., before, during, and/or after the pressure is applied). In some instances, only a portion of the dermal lavage may be used (e.g., needles can be used to create perforations in the skin and the pressure can be applied).

At 62, pigment particles can be suspended within a fluid beneath the surface of the patient's skin. For example, the fluid (e.g., an irrigation fluid) can be introduced to the skin by fluid delivery device 22 and delivered into an irrigation area that includes at least a portion of the tattoo particles (a target region). The irrigation fluid can be a bolus of liquid (e.g., in the range of about 10-50 mL), which is injected into the irrigation area to the target area to be irrigated. In one example, the fluid source can be a reservoir for the irrigation fluid (e.g., an IV bag).

The irrigation fluid can be an exogenous fluid and/or an endogenous fluid. In some instances, the irrigation fluid can be an exogenous fluid, such as a biocompatible fluid, such as saline, sterile water, or the like. In other instances, the irrigation fluid can include an enzyme, natural compound, and/or a pharmaceutical agent. The enzyme or pharmaceutical agent can at least one of lyse cells, make the skin more porous (e.g., by breaking up the tight connections between collagen fibers in the dermis), prevent or inhibit phagocytosis, or otherwise facilitate removal of the irrigation fluid. Examples of enzymes or pharmaceutical agents that can be within the irrigation fluid include hyaluronidase, collegenase, phospholipase, cantherone, colchicine, podophylin, hyaluronic acid, pederine, and sucrose. Examples of a natural compound (e.g., a venom or poison) that can be included in the irrigation fluid include natural or diluted forms of venoms, such as spider, snake, beetle, or bee. In other instances, the enzyme or other chemical agent can be injected before or after the exogenous fluid. For example, the target area can undergo a pre-treatment with histamine, lipase, and/or phospholipase, causing the target area to undergo an inflammatory reaction, which can induce edema and fluid collection/flow to improve draining of the tattoo particles.

In other instances, the fluid can be an endogenous fluid (e.g., derived or obtained from the patient's body). For example, the endogenous fluid can come from local dermal edema. The patient can be injected with a drug, such as a histamine, that is known to cause local dermal edema in the target region of the skin. The fluid can leave the target region through holes or channels, thereby removing the edema and, in the process, washing out the tattoo particles.

At 64, the fluid can be removed (e.g., via passive diffusion, suction, or the like) from the skin with at least a portion (e.g., 10%, 20%, 30%, 40%, 50%, 60%, 70%, 80%, 90%, 99% or more) of the pigment particles (e.g., by fluid removal device 24). In some instances, the tattoo particles and the fluid can be allowed to passively diffuse from the skin. In other instances, suction can be applied continuously or intermittently. Alternatively, suction can be applied in a cyclic manner along with positive pressure to generate a massaging action on the skin and thereby facilitate migration of the exogenous particles to the skin surface. In some instances, the intensity of the suction can be changed over time and thus be applied as a gradient of increasing or decreasing negative pressure.

From the above description, those skilled in the art will perceive improvements, changes and modifications. Such improvements, changes and modifications are within the skill of one in the art and are intended to be covered by the appended claims.

What is claimed is:

1. A method comprising:
applying a pressure to a patient's skin to move pigment particles deeper within the patient's skin so that the pigment particles become less visible,
wherein the pigment particles are created by a tattoo removal process.

2. The method of claim 1, further comprising:
suspending the pigment particles in a fluid beneath a surface of the patient's skin; and
removing the fluid and at least a portion of the pigment particles from the patient's skin.

3. The method of claim 2, wherein the fluid is an endogenous fluid or an exogenous fluid.

4. The method of claim 2, wherein the fluid comprises an enzyme or an agent capable of at least one of lysing cells, making the patient's skin more porous, and preventing or inhibiting phagocytosis.

5. The method of claim 2 further comprising creating the at least one channel through the surface of the patient's skin using a device configured to create the at least one channel.

6. The method of claim 1, wherein the pressure is applied as a shock wave of positive pressure.

7. The method of claim 1, wherein the pressure oscillates between a positive pressure and a negative pressure.

8. The method of claim 7, wherein the oscillating pressure comprises a negative pressure.

9. The method of claim 1, wherein the freeing further comprises using a laser removal procedure to free the ink of the tattoo from the cells.

10. The method of claim 1, wherein the freeing further comprises using a fluid beneath the surface of the patient's skin to free the ink of the tattoo from the cells.

11. The method of claim 1, wherein the applying the pressure further comprises applying vibration to the skin.

12. A system comprising:
a pressure application device configured to apply a pressure to a patient's skin to move freed pigment particles after a procedure that frees ink of a tattoo from cells to create the freed pigment particles within a patient's skin deeper within the patient's skin,
wherein the freed pigment particles become less visible.

13. The system of claim 12 further comprising:
a fluid delivery device configured to deliver a fluid into a patient's skin to suspend ink particles after the ink particles have been freed from a tattoo in the fluid beneath a surface of the patient's skin; and
a fluid removal device configured to facilitate removal of the fluid and the ink particles from the patient's skin.

14. The system of claim 12, wherein the pressure is provided as a shock wave.

15. The system of claim 14, wherein the shock wave is a positive pressure.

16. The system of claim 12, wherein the pressure application device provides an oscillating pressure.

17. The system of claim 16, wherein the oscillating pressure oscillates between a positive pressure and a negative pressure.

18. The system of claim 17, wherein the oscillating pressure comprises a negative pressure.

19. The system of claim 12, wherein the pressure application device provides vibration.

20. The system of claim 12, wherein pressure application device is embodied in a wearable device.
